(19) United States
(12) Plant Patent Application Publication
(10) Pub. No.: US 2002/0088032 P1
(43) Pub. Date: Jul. 4, 2002

(54) Poinsettia plant named "Fiscor Marble"

(75) Inventor: Katharina Zerr, Hohr-Grenzhausen (DE)

Correspondence Address: Foley & Lardner, Suite 500, 3000 K Street, N.W., P.O. Box 25696, Washington, DC, 20007-8696, US

(73) Assignee: FLORFIS AG

(21) Appl. No.: 09/850,054

(22) Filed: May 8, 2001

(30) Foreign Application Priority Data
Nov. 8, 2000 (EP) 2000/1672

(51) Int. Cl. A01H 5/00

(52) U.S. Cl. PLT/303

(57) ABSTRACT

A new and distinct cultivar of Poinsettia plant named ‘Fiscor Marble’, as described and illustrated, and particularly characterized by the combined features of variegated bracts with light-pink center and broad, cream-colored margin; tight, flat, medium-sized inflorescence with weakly lobed bracts; uniform, dark-green foliage, weak to moderately lobed; moderately compact, relatively wide plant habit; and medium early flower response.

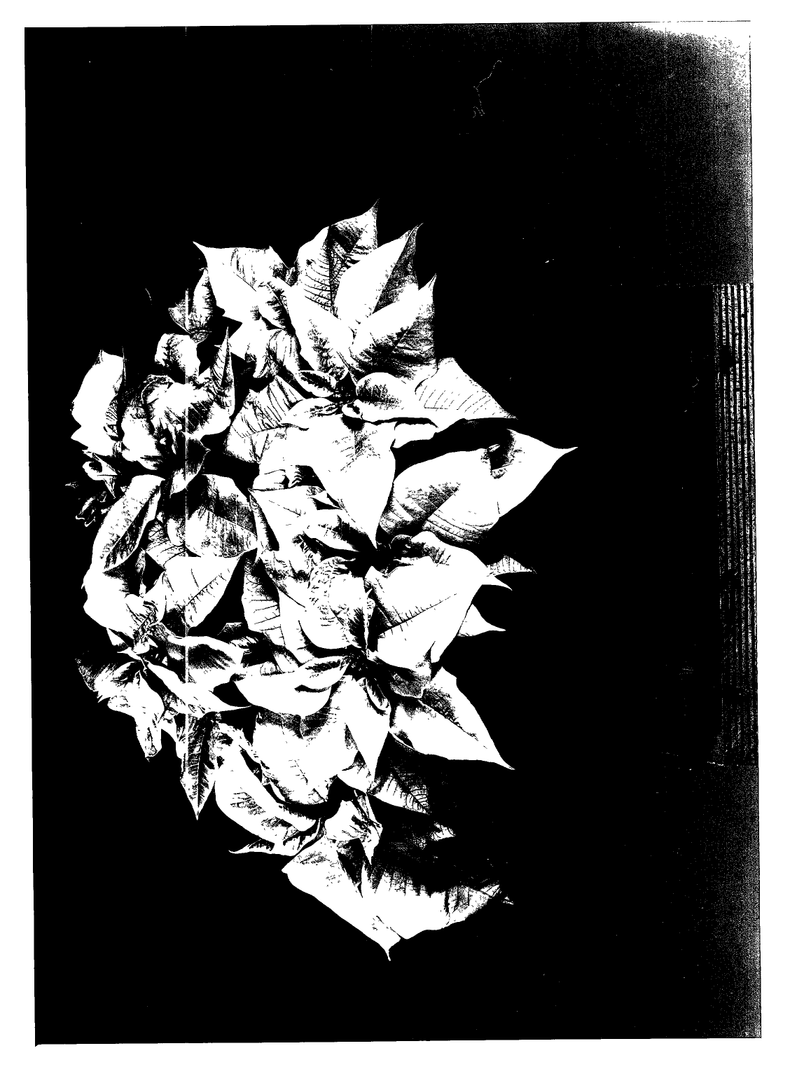

### BACKGROUND OF THE INVENTION

The present invention relates to a new and distinct cultivar of poinsettia plant known by the cultivar name ‘Fiscor Marble’, and botanically known as Euphorbia pulcherrima.

‘Fiscor Marble’ was derived from a naturally occurring mutation in the variety ‘Fiscorosa’ (U.S. Plant Pat No. 10,077). ‘Fiscorosa’ is characterized by pink colored bracts, dark-green foliage, and moderately compact, round and wide plant habit.

The mutation was discovered by Katharina Zerr, the inventor, in a greenhouse in Dorsten, Germany, in the fall of 1997, where small plants had been grown from cuttings of the stock plants of ‘Fiscorosa’ in order to check whether all these plants were still uniform and true to the type.

One plant among the off-spring was completely mutated and displayed variegated, pink and cream colored, bracts. This plant was taken to Hillscheid and left to develop vegetative shoots, which were cut by the inventor used for propagation (first asexual reproduction). The resulting plants were checked for stability of the new characteristics in 1998, and they proved to be uniform,—in contrast to the off-spring of earlier discovered mutants of a similar type. This test was repeated on a larger scale in the fall of 1999.

Horticultural examination (of the clone) starting in 1999 and continuing thereafter has confirmed that the combination of characteristics as herein disclosed for ‘Fiscor Marble’ are firmly fixed and retained through successive generations of asexual reproduction.

### BRIEF DESCRIPTION OF THE INVENTION

The following traits have been repeatedly observed and are determined to be basic characteristics of ‘Fiscor Marble’ which in combination distinguish this Poinsettia as a new and distinct cultivar:

1. variegated bracts with light-pink center and broad, cream-colored margin;

2. tight, flat, medium-sized inflorescence with weakly lobed bracts;

3. uniform, dark-green foliage, weak to moderately lobed;

4. moderately compact, relatively wide plant habit; and

5. medium early flower response.

‘Fiscor Marble’ has not been observed under all possible environmental conditions. The phenotype may vary significantly with variations in environment such as temperature, light intensity, and daylength. The following observations, measurements and comparisons describe plants grown in Hillscheid, Germany, under greenhouse conditions which approximate those generally used in commercial practice.

Of the may commercial cultivars known to the inventor, the most similar in comparison to ‘Fiscor Marble’ are the parent cultivar ‘Fiscorosa’ and the cultivar ‘Freedom Marble’.

In comparison to the pink flowered variety ‘Fiscorosa’, ‘Fiscor Marble’ has variegated, cream and light-pink colored bracts. Additionally, the reddish coloring of the stems and the petioles is weaker with ‘Fiscor Marble’, and the lobing of bracts and leaves is somewhat less distinct.

In comparison to ‘Freedom Marble’, ‘Fiscor Marble’ has similarly or only slightly lighter colored bracts, but its bracts are smaller and have a relatively smooth surface, in contrast to those of ‘Freedom Marble’, which are large and distinctly wrinkled. Furthermore, ‘Fiscor Marble’ has a wider plant habit and does not start flowering as early, but in the medium (mid season) range.

### BRIEF DESCRIPTION OF THE DRAWING

The accompanying color photographic drawing shows typical inflorescence and foliage of ‘Fiscor Marble’, with colors being as true as possible with illustrations of this type. The photograph shows a mature potted plant.

### DETAILED BOTANICAL DESCRIPTION

The plants described were grown in a greenhouse in Hillscheid, Germany, in the fall of 2000. Rooted cuttings had been planted into 12 cm pots on August 10, and plants had been pinched on August 24, leaving 8 nodes. The minimum temperature was 20° C. until the end of September, and about 17-18° C. thereafter. The plants initiated flowers in natural short-day conditions in fall.

Observations and measurements were mainly taken at the beginning of full flowering. In the following description color references are made to The Royal Horticultural Society Colour Chart (RHS). The color values were determined indoors in a north light.

| | |
|---|---|
| CLASSIFICATION: | |
| Botanical: | Euphorbia pulcherrima |
| Commercial: | Poinsettia cv. ‘Fiscor Marble’ |
| PARENTAGE: | Naturally occurring mutation of |
| | ‘Fiscorosa’ |
| PLANT: | |
| Form: | Shrub, self-branching |
| Growth habit: | Moderately compact, medium |
| | height, pinched plants are |
| | bushy and wide in shape |
| Height (above soil line): | 19.5 cm |
| Width: | 45.5 cm |
| Average number of branches: | 7.5 |
| Average number of inflorescence: | 7.0 |
| Stem color: | Dull green, RHS 139 D, usually |
| | without traces of anthocyanin |
| | (reddish color) |
| Rooting: | Medium, about 20-24 days |
| Blooming habit: | Begin under natural short day |
| | conditions in fall: botanically |
| | cyathia open): in early December; |
| | commercially (bracts |
| | colored, marketable): |
| | around December 1 |
| Flowering response time: | About 9 to 9.5 weeks |
| Flowering season: | Up to about 5 weeks |
| Keeping quality (shelf life): | Good, more than 28 days under the |
| | conditions of the trial |
| FOLIAGE: | |
| Shape: | Ovate to broad elliptical, weak |
| | to moderately lobed, rounded to |
| | slightly acute base, and |
| | acuminated tip |
| Margin: | Entire |
| Texture: | Upper surface: smooth and flat, |
| | only weakly veined, |
| | color of veins: RHS 144 B to |
| | RHS 144 C |
| Lower leaf surface: | Flat and smooth, except for the |
| | slightly protruding |
| | midrib and finer side veins, |
| | which are arranged in a palmate |
| | pattern, the vein color is greenish |
| | white, RHS 144 D |
| Leaf blade Size: | 13.0 cm long, 7.8 cm wide |
| Petiole: | 6.5 cm long |
| Color: | Generally dark-green |
| Mature foliage: | Upper surface, RHS 139 A, |
| | under surface, RHS 137 A |
| New foliage: | Upper side: RHS 143 B, |
| | under side: RHS 143 C |
| Leaf petiole Color: | Upper side: light green, |
| | RHS 144 C, lower side: |
| | whitish green, RHS 144 D |
| Aspect: | Almost horizontal |
| DISEASE RESISTANCE: | No special observations made |
| FLOWERING DESCRIPTION: | |

Whole inflorescence with surrounding bracts: medium-sized, tight
and flat with well overlapping bracts, horizontally directed,
diameter: 27.5 cm

| | |
|---|---|
| Bracts: | |
| Number of bracts per inflorescence: | About 10-12 (length over 2 cm) |
| Shape: | Larger bracts are ovate, with |
| | rounded bases, and acuminated |
| | tips, and only weakly lobed, the |
| | smaller, younger bracts are broad |
| | elliptically shaped and usually |
| | without lobes |
| Surface Texture: | Smooth and flat, with the veins |
| | hardly visible on the upper |
| | surface |
| Veins: | As bracts mature, veins create a |
| | more rugose pattern similar |
| | to that of the foliage leaves, |
| | vein color of the lower side is |
| | cream to greenish white, |
| | RHS 159 B to 145 C |
| Bract Color: | Generally bi-colored with light- |
| | pink center and broad, yellowish- |
| | white margin upper surface, |
| | center color RHS 51 C-D, more |
| | intense with younger bracts: |
| | up to RHS 51 B, upper surface, |
| | margin: RHS 4 D to RHS 8 D, |
| | lower surface, middle: |
| | RHS 51 C-D, lower surface, |
| | margin: RHS 4 C to RHS 4 D |
| Size of largest bract: | 13.8 cm long, 9.0 cm wide |
| Petiole length: | 2.2 cm |
| Petiole color: | Whitish- green, weakly brownish |
| | pink infused, approximately |
| | between RHS 144 D to |
| | RHS 159 A, often with a more |
| | intense pink stripe at the sides |
| | of the bract petiole |
| Cyme: | Diameter 15-20 mm, 5-10 cyathia |
| | in a tight cluster |
| Cyathium: | Diameter about 5 mm, color: |
| | light green, RHS 143 C |
| Nectar Cups: | Intense yellow, uniform, |
| | no anthocyanin |
| REPRODUCTIVE ORGANS: | |
| Stamens: | Filaments light pink |
| Pollen: | Moderate, yellow |
| Pistils: | Light pink style and stigma, |
| | 6-lobed stigma |
| Ovaries: | Medium green, RHS 143 A-B, |
| | triangular, 3 ovules |
| Fruit/Seed set: | No seed set observed, |
| | but probably fertile |

I claim:

1. A new and distinct cultivar of Poinsettia plant named ‘Fiscor Marble’, as described and illustrated herein.
